(19) United States
(12) Patent Application Publication
(10) Pub. No.: US 2009/0196841 A1
(43) Pub. Date: Aug. 6, 2009

(54) Color Cosmetics Containing Isoalkane Mixture and Effect Pigments

(75) Inventors: Leila Song, Putnam Valley, NY (US); Betty Aucar, Ossining, NY (US); Tatyana Mytil, Ossining, NY (US)

Correspondence Address: BASF CATALYSTS LLC, 100 CAMPUS DRIVE, FLORHAM PARK, NJ, 07932, US

(73) Assignee: BASF CORPORATION, Florham Park, NJ (US)

(21) Appl. No.: 12/026,079

(22) Filed: Feb. 5, 2008

(51) Int. Cl. A61K 8/31; A61Q 1/06; A61Q 1/10; A61Q 1/02

(52) U.S. Cl. 424/64; 424/63; 424/70.6; 424/70.7

(57) ABSTRACT

The invention provides compositions for color cosmetic formulations comprising a specially formulated isoalkane mixture and effect pigments, and optionally, one or more cosmetically acceptable auxiliary agents, and methods for making the color cosmetics formulations.

### BACKGROUND OF THE INVENTION

Cosmetic formulations are generally defined as compositions suitable for application to the human body. Cosmetic formulations such as creams and lotions are used to moisturize the skin and keep it smooth and supple. Colored cosmetic formulations such as foundation, tanning lotion, correction creams, concealers, body paint, blush, lipstick, lip gloss, mascara, and eye shadow, are used to accent and enhance the hair, skin, eyes, and lips with color. Modern color cosmetic formulations also require effect, e.g., radiance, color correction, artificial tanning, whitening appearance, line-reducing effect, etc., as well as skin conditioning.

The use of effect pigments in color cosmetic formulations aid with the effect and functional appearance issues; however, the formulations often have issues with poor rheology, stability, binding and suspending ability. Many formulations need dispersing agents and avoid using low viscosity ingredients to aid with pigment binding and wetting.

In lipstick or lip gloss, the use of high viscosity oils and waxes delay pigment settling and oiliness. In loose powder, high viscosity emollients are used for pigment finding and wetting. In mascara and hair color, high viscosity components are used to encourage easy and even application, and prevent caking. In general, oils and waxes are used to enhance gloss and emolliency, and also to allow formation of the product into an appropriate shape. For example, an emollient of castor oil or mineral oil can be used to allow the product to be compressed into satisfactory cakes. The use of high viscosity ingredients presents difficulties with tackiness, comedogenicity, and undesired caking.

It is an object of the present invention to provide a colored cosmetic formulation that does not exhibit the formulations problems associated with the non-use of high viscosity emollients. It is an object of the present invention to provide formulations that use a low viscosity emollient that do not have pigment settling issues and that have adequate binding power. It is a further object of the present invention to provide a color cosmetic formulation with high emolliency and humectancy, high fluidity and spreading, high moisturization and hydration, with radiant shine and gloss, softness for a “cashmere feel,” and intense color.

The objects of the present invention are satisfied using effect pigments and a specially formulated isoalkane mixture.

### SUMMARY OF THE INVENTION

The present invention provides compositions for colored cosmetic formulations comprising at least one isoalkane mixture and at least one effect pigment, and optionally, at least one cosmetically acceptable auxiliary agent. The decorative color cosmetics may be any colored cosmetic used on the skin, hair, eyes, or lips, such as concealing sticks, foundation, stage make-up, mascara, eye shadow, hair color, lipsticks, lip gloss, kohl pencils, eye liners, blushers, eyebrow pencils, and cream powders. The specially formulated isoalkane mixture and methods of making it are disclosed in WO2006120003 (PCT/EP2006/004461), which is incorporated herein by reference. Methods for making the color cosmetic formulations using the specially designed isoalkane mixture are also presented.

The specially formulated isoalkane mixture presents certain qualities that are desirable in cosmetics. It has very good dermatological compatibility, imparts a pleasant feel to the hair and the skin, has a good conditioning effect, is compatible with a large number of other active ingredients and auxiliaries, permits adjustment of the rheological properties of the compositions in a wide range (e.g. from liquid to gel-like), and has little to no odor.

The specially formulated isoalkane mixture, which is low viscosity, can be used in color cosmetic formulations to eliminate the typical problems associated with the use of high viscosity emollients, yet provide the desired color effects. To create the color cosmetic formulation, the isoalkane mixture is mixed with the oil phase of the cosmetic formulation. The effect pigment is mixed with either the isoalkane mixture or the appropriate oil or water phase to avoid damage to the fragile effect pigments.

### DETAILED DESCRIPTION OF THE INVENTION

The present invention provides compositions for colored cosmetic formulations comprising at least one isoalkane mixture and at least one effect pigment, and optionally, at least one cosmetically acceptable auxiliary agent. The decorative color cosmetics may be any colored cosmetic used on the skin, hair, eyes, or lips, such as concealing sticks, foundation, stage make-up, mascara, eye shadow, hair color, lipsticks, lip gloss, kohl pencils, eye liners, blushers, eye brow pencils, and cream powders. The form of the compositions may be any form normally used for cosmetics such as cream, emulsion, foam, gel, lotion, milk, mousse, ointment, paste, powder, spray, or suspension. The specially formulated isoalkane mixture and methods of making it are disclosed in WO2006120003 (PCT/EP2006/004461), which is incorporated herein by reference. To create the color cosmetic formulation of the present invention, the isoalkane mixture is mixed with the oil phase of the cosmetic formulation. The effect pigment is mixed with either the isoalkane mixture or the appropriate oil or water phase to avoid damage to the fragile effect pigments.

The isoalkane mixture used in the color cosmetic formulations of the present invention has various qualities that provide its character. The isoalkane mixture has:
i. an average degree of branching as described by the ISO index, which specifies the average number of methyl branches in the olefin fraction;
ii. an ¹H NMR spectrum in the region of a chemical shift δ of from 0.6 to 1.0 ppm, based on tetramethylsilane; a surface integral of from 25 to 70%, based on the total integral surface;
iii. essentially no tert-butyl groups (—C(CH₃)₃), where the fraction of terminal tert-butyl groups is at most 20%, preferably at most 10%, and most preferably at most 5%;
iv. preferably a uniform structure such that the longest continuous carbon chain has essentially or exclusively methyl branches;
v. at least 70% by weight, preferably at least 85% by weight, most preferably at least 95% by weight of alkanes having 8 to 20 carbon atoms;
vi. an oligomeric distribution, i.e., nonuniformity with regard to the number of carbon atoms (and thus the molecular weight) of the alkanes present;
vii. a viscosity, determined in accordance with Brookfield, in the range from 2 to 10 mPas;
viii. kinematic viscosity in the range from 5 to 25 cSt;
ix. a density in the range from 0.7 to 0.82 g/cm³;
x. a refractive index in the range from 1.4 to 1.5; and
xi. a degree of branching B in the range from 0.1 to 0.35, where branching is independent of molecular weight and defined as the number of branches per carbon atom, i.e., B=number of branches/number of carbon atoms; For example, n-octane: 0/8=0, methylheptane: 1/8=0.125, dimethylhexane: 2/8=0.25, squalane: 6/30=0.2.
   Depending on the molecular weight, the isoalkanes are of liquid to oil-like consistency. This isoalkane mixture is commercially available from BASF AG under the tradename LUVITOL LITE.

The colored cosmetic formulations of the present invention comprise from 0.1 to 99.9% by weight of at least one isoalkane mixture and 0.1 to 90% by weight of pigment, and optionally, at least one cosmetically acceptable auxiliary agent. The isoalkane mixture may be used in addition to or in place of other cosmetically acceptable emollients. For example, it may be used in addition to or in place of cosmetically acceptable polymers, waxes, oils, solid and semisolids, such as squalane, petrolatum, or polyisobutane. The amount is the same as that used in standard cosmetic formulations for emollients. If the isoalkane mixture is available as a gel (or at a higher viscosity), more of the isoalkane mixture may be used without creating the problems normally associated with standard high viscosity emollients.

The amount of pigment present in the color cosmetic formulation is dependent on the color cosmetic being created and the final form of the cosmetic. More pigment is used to create higher intensity, or provide higher coverage or correction. One skilled in the art will be able to determine the appropriate amount of pigment to use based upon the desired properties of the colored cosmetic formulation; however, the colored cosmetic formulation comprises from 0.1 to 90% by weight of pigment.

In a cream powder, which goes on as a cream and dries as a powder, the color cosmetic formulation comprises 0.1 to 20% by weight of the isoalkane mixture and 0.1 to 20% by weight of the pigment. In an eye shadow, where the isoalkane mixture acts as a binder and emollient, the color cosmetic formulation comprises 0.1 to 20% by weight of the isoalkane mixture and 0.1 to 90% of the pigment. In a lip balm, which is softer than a lipstick, the color cosmetic formulation comprises 0.1 to 60% by weight of the isoalkane mixture and 0.1 to 40% of the pigment. In a lip gloss, the color cosmetic formulation comprises 0.1 to 80% by weight of the isoalkane mixture and 0.1 to 20% of the pigment. In a loose powder, such as a mineral powder, the color cosmetic formulation comprises 0.1 to 20% by weight of the isoalkane mixture and 0.1 to 90% of the pigment. In a mousse foundation, the color cosmetic formulation comprises 0.1 to 20% by weight of the isoalkane mixture and 0.1 to 40% of the pigment. In body paint, such as eyeliner or a removable tattoo, the color cosmetic formulation comprises 0.1 to 20% by weight of the isoalkane mixture and 0.1 to 90% of the pigment. In a hair or styling gel, the color cosmetic formulation comprises 0.1 to 20% by weight of the isoalkane mixture and 0.1 to 20% of the pigment. In mascara, the color cosmetic formulation comprises 0.1 to 40% by weight of the isoalkane mixture and 0.1 to 40% of the pigment. In a color correction cream, the color cosmetic formulation comprises 0.1 to 30% by weight of the isoalkane mixture and 0.1 to 80% of the pigment. In a tanning cream, the color cosmetic formulation comprises 0.1 to 30% by weight of the isoalkane mixture and 0.1 to 20% of the pigment. In an all purpose stick, such as a concealer of foundation, the color cosmetic formulation comprises 0.1 to 20% by weight of the isoalkane mixture and 0.1 to 40% of the pigment. In a hair putty, the color cosmetic formulation comprises 0.1 to 20% by weight of the isoalkane mixture and 0.1 to 20% of the pigment. In a shampoo or body wash, the color cosmetic formulation comprises 0.1 to 10% by weight of the isoalkane mixture and 0.1 to 10% of the pigment. In each colored cosmetic formulations, one skilled in the art will recognize that the amount of the isoalkane mixture needed may be adjusted based upon the viscosity of the base. One skilled in the art will select the appropriate effect pigment and amount thereof based upon the desired effect, radiance, skin tone, coverage and color. One skilled in the art will also recognize that the amount of pigment used depends upon the viscosity of the base.

Effect pigments, also known as pearlescent pigments or nacreous pigments, are used to impart a pearlescent luster, metallic luster and/or multi-color effect approaching iridescent, to a material. Effect pigments are composed of a plurality of laminar platelets, each of which is coated with one or more reflecting/transmitting layers. Typically, effect pigments are a laminar substrate such as mica or glass flake that has been coated with a metal oxide layer. Pigments of this type were first based on metal oxides, as described in U.S. Pat. Nos. 3,087,828 and 3,087,829. A description of their properties can be found in the Pigment Handbook, Volume I, Second Edition, pp. 829-858, John Wiley & Sons, NY 1988, which is incorporated herein by reference. These pigments exhibit pearl-like luster as a result of reflection and refraction of light, and depending on the thickness of the metal oxide layer, they can also exhibit interference color effects.

Effect pigments can be used in all external and rinse-off applications. Thus, they can be used in hair sprays, face powder, lip coloring, leg-makeup, insect repellent lotions and sprays, mascara, nail enamel, nail enamel remover, perfume, lotion, and shampoos of all types (gel or liquid), shaving cream (concentrate for aerosol, brushless, lathering), skin glosser stick, skin makeup, hair grooming, eye shadow (liquid, pomade, powder, stick, pressed or cream), eye liner, cologne stick, cologne, cologne emollient, bubble bath, body lotion (moisturizing, cleansing, analgesic, astringent), after shave lotion, after bath milk and sunscreen lotion. For a review of cosmetic applications, see Cosmetics: Science and Technology, 2nd Ed., Eds: M. S. Balsam and Edward Sagarin, Wiley-Interscience (1972) and deNavarre, The Chemistry and Science of Cosmetics, 2nd Ed., Vols. 1 and 2 (1962), Van Nostrand Co. Inc., Vols. 3 and 4 (1975), Continental Press, both of which are incorporated herein by reference.

Effect pigments can be manufactured. For example, by precipitating a uniform hydrous metal oxide onto laminar platelets and thereafter calcining the coated platelets to provide metal oxide-coated platelets. The metal oxide in most widespread use is titanium dioxide, followed by iron oxide. Other usable oxides include (but are not limited to) tin, chromium and zirconium oxides as well as mixtures and combinations of oxides. In addition, other colorants such as carmine and ferric ferrocyanide can also be used.

In general, the procedure involves dispersing the particulate (flakes) and combining that dispersion with a precursor, which results in the formation of a titanium oxide or iron oxide precursor coating on the flakes. Usually, the particulate or flakes are dispersed in water, which is preferably distilled. The average particle size of the flakes preferably used can vary from an average of about 3 microns to an average of about 100 microns, although smaller flakes of down to about 1 micron or less or larger flakes of up to 150 microns or more can also be used if desired. The platelets have a thickness of about 0.1 to 10 μm and an aspect ratio (average particle size/thickness) of at least about 10. The concentration of the particulate in the water can vary from about 5 to 60%, although the generally preferred concentrations vary between about 10 and 20%.

An appropriate metal ion source material is added to water/particulate slurry. In the case of titanium, titanyl chloride or titanium tetrachloride is preferably used and in the case of iron, the source material is preferably ferric chloride. The pH of the resulting slurry is maintained at an appropriate level during the addition of the titanium or iron salt by the use of a suitable base such as sodium hydroxide in order to cause precipitation of a titanium dioxide or iron oxide precursor on the particulate. Increasing the thickness gives rise to interference colors. If desired, layers of titanium and iron hydroxide and/or oxide (or other metals) can be deposited sequentially. If necessary to lower the pH, an aqueous acid such as hydrochloric acid can be used. The coated platelets can, if desired, be washed and dried before being calcined to the final effect pigment.

Other coating procedures, such as for example, chemical vapor deposition processes, can also be used.

Optically variable effect pigments may also be used in the present invention. These pigments are constructed with the substrate being coated with a reflecting layer (e.g., silver, gold, aluminum, platinum, palladium, rhodium, ruthenium, osmium, iridium or their alloys) which is overcoated with a low index of refraction material, typically having a refractive index from 1.3 to 2.5, that provides a variable path length for light dependent on the angle of incidence of light impinging thereon (for instance, MgF₂ or SiO₂), which in turn may be overcoated with a third layer selectively transparent to light directed thereon (e.g., silicon, iron oxide, chromium oxide, a mixed metal oxide, titanium dioxide, titanium nitride and aluminum, as well as the same materials as the first layer provided they are sufficiently thin as to be selectively transparent).

The different materials or substrates used to create the effect material may have any morphology including platelet, spherical, cubical, acicular, whiskers, or fibrous. Examples of useful platy materials include platy aluminum oxide, glass, aluminum, mica (natural and synthetic, i.e., fluorphlogopite), bismuth oxychloride, platy iron oxide, platy graphite, platy silica, bronze, stainless steel, natural pearl, boron nitride, silicon dioxide, copper flake, copper alloy flake, zinc flake, zinc alloy flake, zinc oxide, enamel, china clay, metal, plastic, ceramic, and porcelain and the like.

Effect pigments suitable for creating the colored cosmetic formulations of the present invention are also commercially available. The effect pigments include those available from BASF Corporation under the tradenames BIJU, CLOISSONE, DUOCROME, FLAMENCO, and REFLECKS.

For the colored cosmetic formulation of the present invention, one skilled in the art will also select the appropriate cosmetically acceptable auxiliary agent based upon standard cosmetic formulations. The cosmetically acceptable auxiliary agent may be selected from carriers, excipients, emulsifiers, surfactants, preservatives, fragrances, perfume oils, thickeners, polymers, gel formers, dyes, absorption pigments, photoprotective agents, consistency regulators, antioxidants, antifoams, antistats, resins, solvents, solubility promoters, neutralizing agents, stabilizers, sterilizing agents, propellants, drying agents, opacifiers, cosmetically active ingredients, hair polymers, hair and skin conditioners, graft polymers, water-soluble or dispersible silicone-containing polymers, bleaches, care agents, colorants, tinting agents, tanning agents, humectants, refatting agents, collagen, protein hydrolyzates, lipids, emollients and softeners. They may also include tinting agents, tanning agents, bleaches, keratin-hardening substances, antimicrobial active ingredients, photofilter active ingredients, repellant active ingredients, hyperemic substances, keratolytic and keratoplastic substances, antidandruff active ingredients, antiphlogistics, keratinizing substances, active ingredients which act as antioxidants and/or as free-radical scavengers, skin moisturizing or humectant substances, refatting active ingredients, deodorizing active ingredients, sebostatic active ingredients, plant extracts, antierythematous or antiallergic active ingredients and mixtures thereof. The cosmetically acceptable auxiliary agents are commercially available through standard avenues.

In order to illustrate the compositions for colored cosmetic formulations of the present invention, and the methods for making the color cosmetic formulations, various examples are set forth below. It will be appreciated, however, that these are intended to be illustrative and not limiting.

### EXAMPLES

### Example 1A

### Lip Balm with Isoalkane Mixture and without Effect Pigment

| Ingredient | Weight (g) | % w/w |
|---|---|---|
| Isoalkane Mixture (BASF AG) | 12.48 g | 52 |
| Petrolatum | 4.04 g | 17 |
| Beeswax | 1.73 g | 7 |
| Candelilla Wax | 1.33 g | 5.5 |
| 2-Ethylhexyl Ester | 1.05 g | 4 |
| Carnauba wax | 0.73 g | 3 |
| Mineral Oil | 0.66 g | 3 |
| Ozokerite | 0.66 g | 3 |
| Lanolin Alcohol | 0.40 g | 1.5 |
| Trimethylsiloxysilicate | 1.00 g | 4 |

The components were combined in a 250 ml glass beaker and placed on a heating plate. The formulation was mixed at 2500 rpm with a propeller blade while heated with a hot plate to 90° C. It was mixed until uniform for approximately 5 minutes at 90° C. The mix was poured into molds, and then placed in the freezer for 30 min. The resulting formulation was acceptably soft with high gloss, high emolliency and provided a cashmere feel.

### Example 1B

### Lip Balm without Isoalkane Mixture

| Ingredient | Weight | | % w/w |
|---|---|---|---|
| Castor Oil | 11.00 | g | 45.5 |
| Petrolatum | 4.04 | g | 17 |
| Beeswax | 1.73 | g | 7 |
| Candelilla Wax | 1.33 | g | 5.5 |
| 2-Ethylhexyl ester | 1.05 | g | 4 |
| Carnauba wax | 0.73 | g | 3 |
| Mineral oil | 0.66 | g | 3 |
| Ozokerite | 0.66 | g | 3 |
| Cetyl Esters | 0.4 | | 1.5 |
| Trimethylsiloxysilicate | 2.50 | g | 10.5 |

The components were combined in a 250 ml glass beaker and placed on a heating plate. The formulation was mixed at 2500 rpm with a propeller blade while heated with a hot plate to 90° C. It was mixed until uniform for approximately 5 minutes at 90° C. The mix was poured into molds, then placed in the freezer for 30 min. The resulting formulation was very hard.

### Example 1C

### Lip Balm with Isoalkane Mixture and Effect Pigments

| Phase | Ingredients | % w/w |
|---|---|---|
| A | Isoalkane Mixture (BASF AG) | 50.51 |
| | Petrolatum | 16.27 |
| | Beeswax | 7.24 |
| | Euphorbia Cerifera Wax | 5.38 |
| | Copernicia Cerifera Wax | 2.95 |
| | Mineral Oil | 4.05 |
| | Ozokerite | 2.67 |
| | Lanolin Alcohol | 1.62 |
| | Trimethylsiloxysilicate | 4.05 |
| | Ethylhexyl Palmitate | 4.25 |
| B | Effect Pigments (BASF AG) | 1.01 |
| | Preservatives | q.s. |

All of the Phase A ingredients were weighed in a vessel and heated to 85±5° C., then mixed until uniform. The temperature was reduced to 75±5° C., and then the Phase B components were added while mixing. The mix was poured into a mold and cooled in the freezer for 30 min. The resulting formulation was acceptably soft with high gloss, high emolliency and cashmere feel. The color was intense.

### Example 2

### Lip Gloss with Isoalkane Mixture and Effect Pigments

| Phase | Ingredients | % w/w |
|---|---|---|
| A | Isoalkane Mixture (BASF AG) | 61.73 |
| | Dimethicone | 15.98 |
| | Trimethylsiloxysilicate | 16.70 |
| | Fumed Silica | 4.79 |
| B | Effect Pigment (BASF AG) | 0.76 |
| C | Fragrance | 0.04 |

The isoalkane mixture and dimethicone in Phase A were weighed into a mixing vessel and heated to 80±5° C., then mixed until uniform. The trimethylsiloxysilicate and silica in Phase A were added while maintaining temperature then mixed until uniform. The pre-pulverized pigment powders in Phase B were added to Phase A, then mixed until uniform. The mixture was cooled to room temperature and the fragrance added. The mixture was then poured into containers. The resulting formulation was acceptably soft with high gloss, high emolliency and cashmere feel. Although the isoalkane mixture has a lower viscosity than other emollients used in cosmetic formulations, the pigment was still adequately disbursed. The color was intense.

### Example 3

### Lip Gloss with Isoalkane Mixture and Effect Pigments

| Phase | Ingredients | % w/w |
|---|---|---|
| A | C4-24 Alkyl Dimethicone/DivinylDimethicone | 15.00 |
| | Crosspolymer Dimethicone (DM) | |
| | Methylheptylisostearate | 12.00 |
| | C4-24 Alkyl Dimethicone/DivinylDimethicone | 56.60 |
| | Crosspolymer Dimethicone (DM-6) | |
| | Vitamin E Acetate | 0.20 |
| | Preservatives | q.s. |
| | Fragrance | 0.20 |
| B | Effect Pigment Mixture (BASF AG) | 6.00 |
| | Isoalkane Mixture (BASF AG) | 10.00 |

The Phase A Ingredients were added to a vessel, and then homogenized until uniform. The Phase B ingredients were gently mixed to avoid damage to the effect pigment. The premixed Phase B ingredients were added to the premixed Phase A ingredients. All of the ingredients were mixed until uniform. The mixture was poured into appropriate containers. The resulting formulation was acceptably soft with high gloss, high emolliency and cashmere feel. The pigment was still adequately disbursed. The color was intense.

### Example 4

### Mousse Foundation with Isoalkane Mixture and Effect Pigment

| Phase | Ingredients | % w/w |
|---|---|---|
| A | Dimethicone (and) Cetearyl Dimethicone Crosspolymer | 48.01 |
| | Caprylyl Methicone | 22.94 |
| | Isoalkane Mixture(BASF AG) | 2.20 |
| B | Sunscreen (Titanium Dioxide) | 14.11 |
| | Sunscreen (Zinc Oxide) | 1.68 |
| C | Effect Pigment Mixture (BASF AG) | 11.06 |
| D | Fragrance | q.s. |

The Phase A ingredients were added to a vessel, and then mixed until uniform. The pre-pulverized Phase B ingredients were added to Phase A, then mixed until uniform. The Phase C ingredients were added to the Phase A-B mixture, and then mixed until uniform. The Phase D ingredients were added to the Phase A-B-C mixture, and then mixed until uniform. The mixture was homogenized as necessary. The resulting formulation was acceptably soft with high gloss, high emolliency and cashmere feel. The color was intense.

### Example 5

### Organic Mineral Powder with Isoalkane Mixture and Effect Pigment

| Phase | Ingredients | % w/w |
|---|---|---|
| A | Effect Pigment 1 (BASF AG) | 63.00 |
| | Sunscreen (Titanium Dioxide) | 3.00 |
| | Sunscreen (Zinc Oxide) | 2.00 |
| | Dimethicone/vinylDimethicone Crosspolymer (and) | 2.00 |
| | Silica | |
| | Preservatives | q.s. |
| | Antioxidants | q.s. |
| | Effect Pigment 2 (BASF AG) | 12.50 |
| | Effect Pigment 3 (BASF AG) | 12.50 |
| B | Isoalkane Mixture(BASF AG) | 2.50 |
| | Octyldodecyl Neopentanoate | 2.50 |

Phase A ingredients were dry blended in appropriate dry blending/dispersing equipment. Phase B ingredients were combined and sprayed into Phase A. The mixture was pulverized until uniform. The formulation was packaged into appropriate containers. The resulting formulation had high gloss, high emolliency and cashmere feel. The color was intense.

### Example 6

### Loose Powder Eyeshadow with Isoalkane Mixture and Effect Pigment

| Ingredients | % w/w |
|---|---|
| Cyclopentasiloxane (and) C30-45 Alkyl Cetearyl Dimethicone | 14.86 |
| Copolymer | |
| Isoalkane Mixture (BASF AG) | 5.61 |
| Effect Pigment 1 (BASF AG) | 42.10 |
| Effect Pigment 2 (BASF AG) | 37.43 |
| Preservatives | q.s. |

All ingredients were added to a vessel and mixed until uniform. The powders were sieved through a screen, and then packaged into containers. The resulting formulation had high gloss, high emolliency and cashmere feel. The color was intense.

### Example 7

### Pressed Powder Eyeshadow with and without Isoalkane Mixture

| | | | % w/w |
|---|---|---|---|
| | | % w/w | Deep |
| Phase | Ingredients | Light Color | Color |
| A | Mearltalc TCA (talc lauryl lysine) | 43.00 | 42.13 |
| | Zinc Stearate | 4 | 4 |
| | Boron Nitride | 2 | 2 |
| | Effect Pigment (BASF AG) | 3 | 3 |
| | Preservatives | .50 | .50 |
| B | Effect Pigment 1 (BASF AG) | 35 | 35 |
| | Effect Pigment 2 (BASF AG) | 5 | 5 |
| C | Isoalkane Mixture(BASF AG) | 5.5 | — |
| | Squalane | — | 6.37 |
| | Vitamin E | 0.50 | 0.50 |
| | Dimethicone (and) Cetearyl Dimethicone | 1.50 | 1.50 |
| | Crosspolymer | | |

The Phase A ingredients were thoroughly blended and dispersed in appropriate dry blending/dispersing equipment. Phase B ingredients were added into a support vessel. The mixture was heated and mixed until uniform. The Phase B ingredients were sprayed into premixed Phase A ingredients and blending continued. The ingredients were pulverized and returned to the blender. The Phase C ingredients were added to the Phase A-B mixture and mixed until uniform. The formulation was pressed into an appropriate container. Both pressed powder eye shadows passed the drop test. The eye shadow with the isoalkane mixture provided a smoother spread and silkier feel.

### Example 8

### Hair Putty with Isoalkane Mixture and Effect Pigment

| Phase | Ingredients | % w/w |
|---|---|---|
| A | Cyclopentasiloxafle (and) PEG/PPG-20/15 Dimethicone | 2.38 |
| | Phenyl Trimethicone | 4.76 |
| | Isoalkane Mixture (BASF AG) | 0.95 |
| | Euphorbia Cerifera Wax | 7.14 |
| | Beeswax | 11.89 |
| | PEG-20/PPG-23 Dimethicone | 2.38 |
| B | Stearalkonium Hectonte (and) Isopropyl Myristate (and) | 27.59 |
| | Propylene Carbonate | |
| | Cyclopentasiloxane (and) C30-45 Alkyl Cetearyl | 38.06 |
| | Dimethicone | |
| | Copolymer | |
| | Effect Pigment 1 (BASF AG) | 0.57 |
| | Preservatives | q.s. |
| C | Effect Pigment 2 (BASF AG) | 2.57 |
| | Polysorbate 80 | 1.71 |

The Phase A ingredients were added to a vessel and heated to 85±5° C., then mixed until uniform. The Phase B ingredients were added one at a time to Phase A while maintaining temperature and mixing until uniform. The effect pigment was predisbursed into Polysorbate 80 in Phase C at 500 rpm and heated to 85±5° C. Phase C was added to Phase AB, and mixed until uniform. The resulting formulation was poured into containers. The resulting formulation had high gloss, high emolliency and cashmere feel. The color was intense.

### Example 9

### Tanning Cream with Isoalkane Mixture and Effect Pigment

| Phase | Ingredients | % w/w |
|---|---|---|
| A | DI Water (q.s. to 100%) | 57.65 |
| | Disodium EDTA | 0.05 |
| | Xanthan Gum | 0.05 |
| | Carbomer | 0.50 |
| | Acrylates/C10-30 Alkyl Acrylate Crosspolymer | 0.20 |
| A′ | Moisturizer | 3.00 |
| | Butylene Glycol | 2.00 |
| | Isoalkane Mixture (BASF AG) | 2.00 |
| B | Cetyl Alcohol | 2.00 |
| | Glycol Stearate | 2.50 |
| | Squalane | 1.50 |
| | Shea Butter | 2.00 |
| | PEG-100 Stearate | 2.50 |
| | Ethylhexyl Methoxycinnamate | 4.00 |
| | Benzophenone-3 | 3.00 |
| C | Dimethicone | 1.50 |
| | Cyclopentasiloxane (and) Cyclohexasiloxane | 0.40 |
| | Cyclohexasiloxane (and) Cyclopentasiloxane | 3.00 |
| D | Phenoxyethanol (and) Chlorphenesin (and) Propylene | 1.00 |
| | Glycol (and) Sorbic Acid | |
| E | Dihydroxyacetone | 2.00 |
| F | DI Water | 2.00 |
| | Triethanolamine | 0.40 |
| G | DI Water | 5.00 |
| | Effect Pigment Mixture (BASF AG) | 1.75 |

The Phase A ingredients were premixed, one by one, until uniform. The Phase A′ ingredients were added, one by one, to Phase A, with mixing between additions. The heating to 75-80° C. was started. The Phase B ingredients were combined in a secondary vessel and heated to 75-80° C. until completely melted. The Phase A ingredients were added to Phase B while homomixing. The ingredients were mixed until uniform, then cooled to 45-50° C. The Phase C ingredients were added to the Phase A-B mixture at 45-50° C. and mixed until uniform. The cool down was continued. Phase D was added to the Phase A-B-C mixture at 30-35° C. and mixed until uniform. The cool down was continued. The Phase E ingredients were added to the main vessel, then homogenized well. (The ingredients were checked to make sure no powder lumps were present). The premixed Phase F ingredients were added to the batch and mixed until uniform. The premixed Phase G ingredients were added to the batch and homogenized until uniform.

### Example 10

### All Purpose Stick with Isoalkane Mixture and Effect Pigment

| Phase | Ingredients | % w/w |
|---|---|---|
| A | Octyldodecyl Neopentanoate | 47.27 |
| | Effect Pigment Mixture (BASF AG) | 15.00 |
| | Isoalkane Mixture (BASF AG) | 3.13 |
| B | Acetylated Glycol Stearate | 22.00 |
| | Silica | 1.50 |
| C | Simmondsia Chinensis (Jojoba) Seed Oil | 7.50 |
| | Stearic Acid | 3.60 |
| | Preservatives | q.s. |
| | Antioxidants | q.s. |

In a secondary vessel, the pigment was added to Phase A ingredients with mixing under low shear. In the main vessel, Phase B ingredients were mixed. When the Phase B ingredients were melted and uniform, the Phase A ingredients were added. Heating was continued at 80-85° C. with mixing. The Phase C ingredients were slowly added to the Phase A-B mixture. The formulation was poured into a mold, and packaged into appropriate container. The resulting formulation had high gloss, high emolliency and cashmere feel. The color was intense.

### Example 11

### Color Correction Cream with Isoalkane Mixture and Effect Pigment

| | | % |
|---|---|---|
| Phase | Ingredients | w/w |
| A | Isoalkane Mixture (BASF AG) | 20.00 |
| | Acrylates/C10-30 Alkyl Acrylate Crosspolymer | 0.20 |
| | (Pemulen TR-1) | |
| | Preservatives | q.s. |
| | Caprylic/Capric Triglyceride (and) Sodium Acrylates | 0.90 |
| | Copolymer) | |
| B | Ceteareth-25 | 4.00 |
| | Propylene Glycol | 5.00 |
| | Benzophenone-3 | 3.00 |
| | DI Water (q.s. to 100%) | 56.60 |
| C | Fragrance | 0.20 |
| | Sodium Hydroxide (20% aqueous solution) (q.s. to | q.s. |
| | pH 6.0) | |
| D | DI Water | 5.00 |
| | Effect Pigment (BASF AG) | 5.00 |
| | Colorant (0.50% aqueous solution) | 0.10 |

The Phase A components were mixed. The Phase B components were mixed and dissolved. Phase B was stirred slowly with Phase A. The phases were homogenized for a short time. The mixture was neutralized with Phase C and stirred until homogeneous. Phase D was premixed and added to the Phase A-B-C mixture, then homogenized until uniform. The resulting formulation had high gloss, high emolliency and cashmere feel. The color was intense.

### Example 12

### Cream Powder with Isoalkane Mixture and Effect Pigment

| Phase | Ingredients | % w/w |
|---|---|---|
| A | Cyclopentasiloxane (and) PEG/PPG-20/15 Dimethicone | 23.16 |
| | Cyclopentasiloxane (and) C30-45 Alkyl Cetearyl | 49.61 |
| | Dimethicone Copolymer | |
| | Trimethylsiloxysilicate | 0.66 |
| | Caprylyl Methicone | 0.66 |
| | PEG-20/PPG-23 Dimethicone | 3.31 |
| B | Cyclopentasiloxane | 3.31 |
| | Isoalkane Mixture (BASF AG) | 12.00 |
| | Chromium Hydroxide Greens | 0.69 |
| | Iron Oxides | 0.03 |
| | Sunscreen (TiO2) | 3.31 |
| | Effect Pigment Mixture (BASF AG) | 3.23 |
| | Fragrance | 0.03 |

Phase A ingredients were added to a vessel, and then mixed until uniform. The Phase B colorants were pre-pulverized separately, and gradually added in the order listed. The mixture was mixed until uniform. The resulting formulation had high gloss, high emolliency and cashmere feel. The color was intense.

### Example 13

### Body Paint with Isoalkane Mixture and Effect Pigment

| Phase | Ingredients | % w/w |
|---|---|---|
| A | 3-Ethylheptamethyltrisiloxane | 60.99 |
| | Isoalkane Mixture (BASF AG) | 14.90 |
| B | Trimethylsiloxysilicate | 13.55 |
| | Fumed Silica | 4.47 |
| C | Effect Pigment (BASF AG) | 6.09 |
| | Fragrance | q.s. |

The 3-Ethylheptamethyltrisiloxane and isoalkane mixture was added to a mixing vessel, heated to 85+5° C., and mixed until uniform. The Phase B ingredients were added one at a time to Phase A with constant stirring. The ingredients were mixed until uniform. The mixture was cooled to room temperature. Phase C was added and mixed until uniform. Solvent loss was compensated for as necessary. The resulting formulation had high gloss, high emolliency and cashmere feel. The color was intense.

### Example 14

### Hair Styling Gel with Isoalkane Mixture and Effect Pigment

| Phase | Ingredients | % w/w |
|---|---|---|
| A | DI Water (q.s. to 100%) | 69.65 |
| | Tetrasodium EDTA | 0.05 |
| | Panthenol | 0.50 |
| B | Isoalkane Mixture (BASF AG) | 2.50 |
| | PEG 90M | 0.20 |
| C | PEG/PPG-18/18 Dimethicone | 0.50 |
| | Polysorbate 20 | 1.00 |
| | Caprylic/Capric Triglyceride (and) Sodium Acrylates | 5.00 |
| | Copolymer | |
| | Fragrance | 0.10 |
| D | VP/Methyacrylamaide/VinylImidazidecopolymer | 10.00 |
| | Polyquaternium-68 | 2.00 |
| E | Preservatives | q.s. |
| | DI Water | 5.00 |
| | Effect Pigment (BASF AG) | 1.00 |
| | Colorant (0.50% aqueous solution) | 2.50 |

The Phase A ingredients were mixed one at a time until completely homogenous. The Phase B ingredients were pre-mixed in a different vessel, and added to Phase A. The ingredients of Phase C were added to the Phase A-B mixture, one at a time, and mixing continued. Phase D ingredients were added to the Phase A-B-C mixture and mixing continued until homogeneous. The Phase E ingredients were added to Phase A-B-C-D mixture. The resulting formulation had high gloss, high emolliency and cashmere feel. The color was intense.

### Example 15

### Mascara with Isoalkane Mixture and Effect Pigment

| Phase | Ingredients | % wt |
|---|---|---|
| A. | Water (q.s. to 100%) | 47.50 |
| | Triethanolamine | 2.00 |
| | Isoalkane Mixture (BASF AG) | 15.00 |
| B | Effect Pigment (BASF AG) | 10.2 |
| | Iron Oxides | 1.80 |
| C | Candelilla Wax | 12.00 |
| | Stearic Acid | 4.50 |
| | Isostearic Acid | 4.50 |
| | Preservatives (oil soluble) | q.s. |
| D | Water | 2.50 |
| | Preservatives (water soluble) | q.s. |

Phase A ingredients were added to the main vessel. Phase B ingredients were added to Phase A with agitation, and the temperature raised to 75±3° C. Phase C ingredients were added to the support vessel and mixed while raising temperature to 80±3° C. When the water and oil phases were both at 75±3° C., Phase C was added to the Phase A-B mixture and the sweep agitation maintained. The batch was cooled to 50° C. with constant agitation, and the pre-mixed Phase D solution was added to the Phase A-B-C mixture. Stirring was continued and the mixture cooled to 40° C. (The batch may be filled below this temperature.) The resulting formulation had high gloss, high emolliency and cashmere feel. The color was intense.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

We claim:

1. A colored cosmetic formulation comprising at least one isoalkane mixture and at least one effect pigment, and optionally, at least one cosmetically acceptable auxiliary agent.

2. The colored cosmetic formulation of claim 1 comprising from 0.1 to 99.9% by weight of at least one isoalkane mixture and 0.1 to 90% by weight of at least one effect pigment.

3. The colored cosmetic formulation of claim 1 wherein the isoalkane mixture replaces a standard cosmetic emollient.

4. The colored cosmetic formulation of claim 1 wherein the isoalkane mixture is used in addition to a standard cosmetic emollient.

5. The colored cosmetic formulation of claim 1 wherein the color cosmetic is selected from the group consisting of a cream powder, eye shadow, lipstick, lip gloss, lip balm, mineral powder, mousse foundation, body paint, hair gel, mascara, correction cream, tanning cream, all purpose stick, hair putty, hair color, shampoo, and foundation.

6. The colored cosmetic formulation of claim 1 in the form of a cream, emulsion, foam, gel, lotion, milk, mousse, ointment, paste, powder, spray, or suspension.

7. The colored cosmetic formulation of claim 1 having a characteristic of at least one of high emolliency, high humectancy, high fluidity, high spreading, high moisturization, high hydration, radiant shine, high gloss, a cashmere feel, and intense color.

8. The colored cosmetic formulation of claim 5 wherein the color cosmetic is a cream powder comprising 0.1 to 20% by weight of the isoalkane mixture and 0.1 to 20% by weight of the pigment.

9. The colored cosmetic formulation of claim 5 wherein the color cosmetic is an eyeshadow comprising 0.1 to 20% by weight of the isoalkane mixture and 0.1 to 90% of the pigment.

10. The colored cosmetic formulation of claim 5 wherein the color cosmetic is a lip balm comprising 0.1 to 60% by weight of the isoalkane mixture and 0.1 to 40% of the pigment.

11. The colored cosmetic formulation of claim 5 wherein the color cosmetic is a lip gloss comprising 0.1 to 80% by weight of the isoalkane mixture and 0.1 to 20% of the pigment.

12. The colored cosmetic formulation of claim 5 wherein the color cosmetic is a mineral powder comprising 0.1 to 20% by weight of the isoalkane mixture and 0.1 to 90% of the pigment.

13. The colored cosmetic formulation of claim 5 wherein the color cosmetic is a mousse foundation comprising 0.1 to 20% by weight of the isoalkane mixture and 0.1 to 40% of the pigment.

14. The colored cosmetic formulation of claim 5 wherein the color cosmetic is a body paint comprising 0.1 to 20% by weight of the isoalkane mixture and 0.1 to 90% of the pigment

15. The colored cosmetic formulation of claim 1 wherein the color cosmetic is a hair gel comprising 0.1 to 20% by weight of at least one isoalkane mixture and 0.1 to 20% of at least one pigment.

16. The colored cosmetic formulation of claim 5 wherein the color cosmetic is mascara comprising 0.1 to 40% by weight of at least one isoalkane mixture and 0.1 to 40% of at least one pigment.

17. The colored cosmetic formulation of claim 5 wherein the color cosmetic is a color correction cream comprising 0.1 to 30% by weight of the isoalkane mixture and 0.1 to 80% of the pigment.

18. The colored cosmetic formulation of claim 5 wherein the color cosmetic is a tanning cream comprising 0.1 to 30% by weight of the isoalkane mixture and 0.1 to 20% of the pigment.

19. The colored cosmetic formulation of claim 5 wherein the color cosmetic is an all purpose stick comprising 0.1 to 20% by weight of the isoalkane mixture and 0.1 to 40% of the pigment.

20. The colored cosmetic formulation of claim 5 wherein the color cosmetic is a hair putty comprising 0.1 to 20% by weight of the isoalkane mixture and 0.1 to 20% of the pigment.

21. The colored cosmetic formulation of claim 5 wherein the color cosmetic is a shampoo comprising 0.1 to 10% by weight of the isoalkane mixture and 0.1 to 10% of the pigment.

22. The colored cosmetic formulation of claim 1 wherein the cosmetically acceptable auxiliary agent is selected from the group consisting of carriers, excipients, emulsifiers, surfactants, preservatives, fragrances, perfume oils, thickeners, polymers, gel formers, dyes, absorption pigments, photoprotective agents, consistency regulators, antioxidants, antifoams, antistats, resins, solvents, solubility promoters, neutralizing agents, stabilizers, sterilizing agents, propellants, drying agents, opacifiers, cosmetically active ingredients, hair polymers, hair and skin conditioners, graft polymers, water-soluble or dispersible silicone-containing polymers, bleaches, care agents, colorants, tinting agents, tanning agents, humectants, refatting agents, collagen, protein hydrolyzates, lipids, emollients and softeners, tinting agents, tanning agents, bleaches, keratin-hardening substances, antimicrobial active ingredients, photofilter active ingredients, repellant active ingredients, hyperemic substances, keratolytic and keratoplastic substances, antidandruff active ingredients, antiphlogistics, keratinizing substances, active ingredients which act as antioxidants and/or as free-radical scavengers, skin moisturizing or humectants substances, refatting active ingredients, deodorizing active ingredients, sebostatic active ingredients, plant extracts, antierythematous or antiallergic active ingredients and mixtures thereof.

23. A method of making a color cosmetic formulation comprising mixing the isoalkane mixture with the oil phase of the cosmetic formulation; and mixing the effect pigment with the appropriate oil or water phase.
